(19) United States
(12) Patent Application Publication
(10) Pub. No.: US 2021/0056511 A1
(43) Pub. Date: Feb. 25, 2021

(54) Bar Dash - On Demand Bar and Restaurant Staffing Service

(71) Applicants: Jacqueline Underwood, Antioch, CA (US); Nicole Guest, Berkeley, CA (US)

(72) Inventors: Jacqueline Underwood, Antioch, CA (US); Nicole Guest, Berkeley, CA (US)

(21) Appl. No.: 16/550,181

(22) Filed: Aug. 24, 2019

(51) Int. Cl. G06Q 10/10; G16H 40/20

(52) U.S. Cl. CPC G06Q 10/1053; G16H 40/20

(57) ABSTRACT

Systems and methods are disclosed herein for computer-aided method of hospitality industry recruitment system. The method allows applicants to upload their profile and availability status and employers can select the employee based in their requirements. The recruiter can select the applicant, designate the job, get the job completed and provide the required dues using the platform. The system provides online tool to display the available resources to recruiters, their details, billing platform and connection with banks for secured transactions. Furthermore, the clients are rated based on aspects of their performance such as hard work, reliability and overall quality of the job. This information is then permanently saved within the interface for creation of realistic ratings.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field of the Invention

The present invention provides a method of creating independent workers database who can provide instant services on immediate requirement. More particularly the current invention is a complete automated process of hiring job seekers instantly and is performed electronically via an intranet on smart devices.

### Description of the Related Art

Many people today are working in industries outside the box. There are countless day to day service-based tasks, which make up a wide variety of jobs in our community. Yet, these independent contractors and employees are bound to word of mouth testimony, verifying that they are who they say they are, and that their talent is to the standard that they're sharing with the world. There is no efficient system particularly for restaurants, bars or hotels is still yet developed where employee need on day to day basis can be met.

There are multiple employee recruitment systems that have been developed. For instance, an employee recruiting system and method bearing U.S. Pat. No. 8,200,584B2 is issued to Carl E Brickman, Jr. The patent discloses a system for advertising an employment opportunity and efficiently providing information needed for a prospective employee to make a holistic assessment of the opportunity includes a job posting made accessible to potential candidates via a computer network or computer readable media. The job posting is comprised of a plurality of sections, with each section being devoted to one or more specific topics. The sections are accessible from an intuitive main menu. The sections provide an orderly arrangement of details concerning the employment position, significant co-workers, schools, housing, the community, nearby universities, an interview itinerary and links to local websites of interest, as well as views of residents (e.g., community leaders) concerning the community. The information may be presented in text, graphics, animation, audio and/or video formats. Other features include an email to a friend feature, a link to ask questions, a link for an application, and instructions and/or a link for submitting a resume. A network-accessible wizard automates creation of the posting.

Another patent on Electronic employee selection systems and methods bearing US patent 2,005,0246299A1 is issued to Kronos Talent Management Inc. The patent discloses an automated employee selection system can use a variety of techniques to provide information for assisting in selection of employees. For example, pre-hire and post-hire information can be collected electronically and used to build an artificial-intelligence based model. The model can then be used to predict a desired job performance criterion (e.g., tenure, number of accidents, sales level, or the like) for new applicants. A wide variety of features can be supported, such as electronic reporting. Pre-hire information identified as ineffective can be removed from a collected pre-hire information. For example, ineffective questions can be identified and removed from a job application. New items can be added and their effectiveness tested. As a result, a system can exhibit adaptive learning and maintain or increase effectiveness even under changing conditions.

Another patent on Match-based employment system and method bearing U.S. Pat. No. 7,805,382B2 is issued to CITIZENS BANK OF PENNSYLVANIA. The patent discloses in one embodiment of the present invention, a match-based employment system and method of operation are provided. The match-based employment system collects a plurality of employer seeker and employee seeker profiles, bi-directionally matches the employer seeker and said employee seeker profiles and displays at least a portion of the results to an employer seeker or an employee seeker. The match-based employment system can also order the bi-directional matching results based on a bi-directional match score and display the bi-directional matching results according to the ordering. The match-based employment system can also perform the matching such that approximately 70% of a matching score depends upon the quality of the match between employee seeker desires and employment seeker attributes and approximately 30% of the matching score depends upon the quality of the match between employment seeker desires and employee seeker attributes.

An Employee recruiting systems and methods bearing U.S. patent 2,006,010,0919A1 is issued to Paul Levine. The patent discloses methods and systems for employment recruiting are provided. In one aspect, a remote call center is used to make initial contact with the candidates to obtain information regarding their placeability and urgency in changing jobs, and to provide an initial screening (107), and a local candidate specialist may then personally interview the candidate. In another aspect, compensation is set for recruiters by adjusting a base compensation based on individual and group performance. In another aspect, candidates are assigned to candidate specialists, and client employers are assigned to client specialists. In another aspect, job candidates and job openings are independently obtained so that candidates will be available immediately to fill the openings. In another aspect, in a team-oriented method of matching candidates with jobs, candidates are matched to jobs by corporation among candidate specialists and client specialists. In another aspect, candidates are matched with jobs using candidate and employer ratings.

Another patent on system and method for recruiting employees bearing US patent 2,002,0156674A1 is issued to International Business Machines Corp. The patent discloses an efficient operation of an intermediate enterprise is achieved by disclosing, at all times, jobs for which employees are requested and the application status of applicants for those jobs. A network-connected server provides information in response to accesses by member clients via the network. The server includes: a job offer information database for storing job offer information that is managed for each job; an application acceptance unit for accepting applications from client terminals for jobs included in the job offer information; and a job offer information/application status generating unit for presenting to a new applicant not only the job offer information but also information concerning previous applicants who applied for each job included in the job offer information.

Another patent on system and method for recruiting, tracking, measuring, and improving applicants, candidates, and any resources qualifications, expertise, and feedback bearing US patent 2,011,0276507A1 is issued to Matthew Carl O'Malley. The patent discloses a system and method for locating resources and providing recruitment information. Including a memory device for storing the information regarding a at least one resource, a at least one resource criteria, a at least one resource match, and a at least one processing device for processing information regarding the information, criteria, resource and match. In one aspect, the processing device utilizes information regarding a at least one job opening, an applicant's contract, an interviewer's available date, an interviewer available time, and a interview schedule, stored in the memory device, and further wherein the processing device generates a message containing information regarding at least one of a job opening, an interview schedule, an interviewer, an applicant's contract, a date and a time, wherein the message is responsive to the job interview request, and a transmitter for

There are multiple solutions that have been presented in prior art. Within the last decade the advancement in employee recruitment system has been greatly reshaped. Looking at the prior art in a similar field, the improvement in effective utilization of technology and expansion in their utility for every type of workforce is greatly challenged. Most of these systems are made for a particular working scenario or environment. No such system has been found which works independently, is trustworthy, specifically for the day to day service-based workers, where they can render their services on their availability basis and the recruiters also have the opportunity to hiring workforce as per their variable requirements.

The current invention proposes a system which works to ensure that even if someone loses their job or is faced with an unfortunate situation or is looking to start a job can find the available slots easily. This guaranteed database of hiring will be equally beneficial for the recruiters where they can use the proposed system to contact jobseekers or post jobs for jobseekers to fill a shift.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is therefore the purpose of the invention to alleviate at least to some extent one or more of the aforementioned problems of the prior art and/or to provide the relevant public with a suitable alternative thereto having relative advantages.

The primary object of the invention is related to the provision of guaranteed active database of employees available related to bars, hotel and restaurant service-based industry.

It is also the objective of the invention to give a system which ensures that the recruiters can get the required resource earliest possible within their business locality.

It is further the objective of the invention to give a system which works to ensure that even if someone is actively looking for a job or loses the job or is faced with an unfortunate situation regarding their work or have no contacts can sign up with the proposed system and get the job offers while sitting at home.

It is also the objective of the invention to provide a system which is particularly developed for service-based hotel staff including but not limited to Chefs, all kitchen positions, all back end and front-end rolls in a bar, restaurant, hotel, casino, country clubs or private events service jobs.

It is moreover the objective of the invention to provide a system which works on multiple indicators including but not limited to employee availability, proximity to recruiter's work place, education, prior experience and other indicators as selected by recruiter.

It is further the objective of the invention to provide a reliable system which works efficiently and has a strong network system to achieve maximum output and is compatible for all type of Android and iOS systems.

It is moreover the objective of the invention to provide a system which is suitable for shift-based hiring, day to day hiring or contractual hiring.

It is further the objective of the invention to provide a system which is easy to use, easy to implement and provides an advance methodology of employing, service-based industry available resources.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DESCRIPTION OF THE DRAWINGS

Please include description of drawings as follows:

FIG. 1 is flow diagram showing the process of proposed system hiring for candidate, consistent with the exemplary embodiments of the present invention

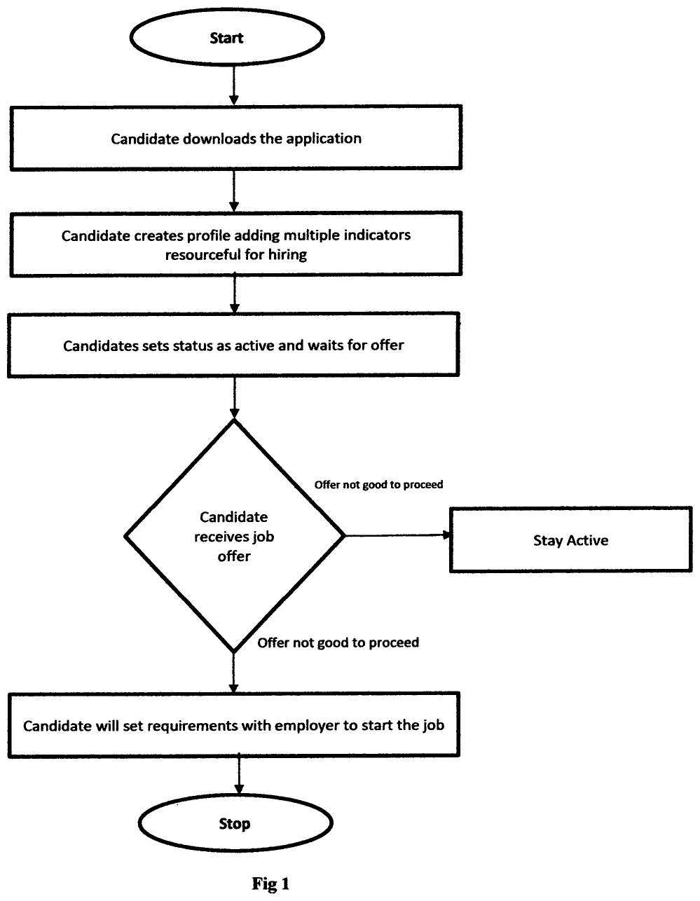

FIG. 2 is flow diagram showing operation of the invention once candidate is contacted by the recruiter, consistent with the exemplary embodiments of the present invention.

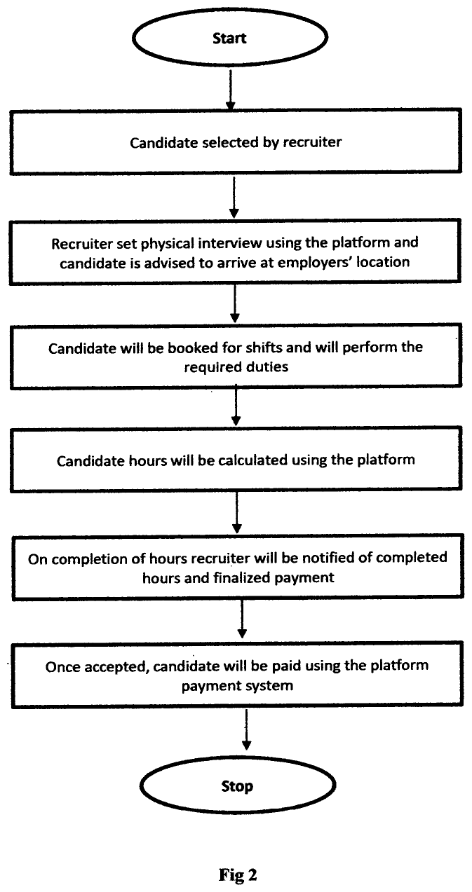

FIG. 3 is flow diagram showing the process of final hiring steps for the candidate as per preferred embodiments of the invention.

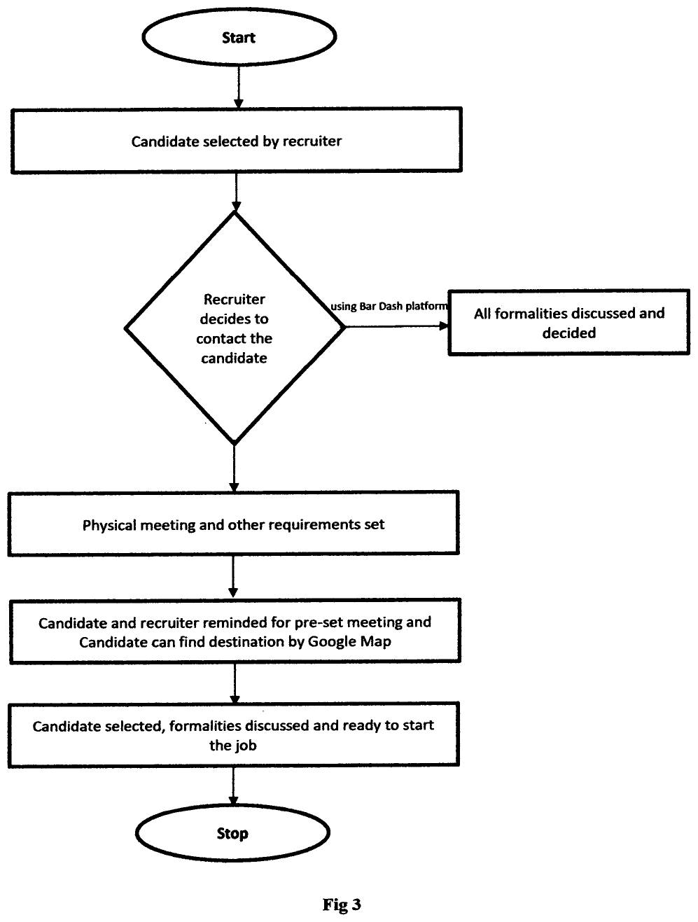

FIG. 4 is flow diagram showing the process of proposed system for the recruiter, consistent with the exemplary embodiments of the present invention.

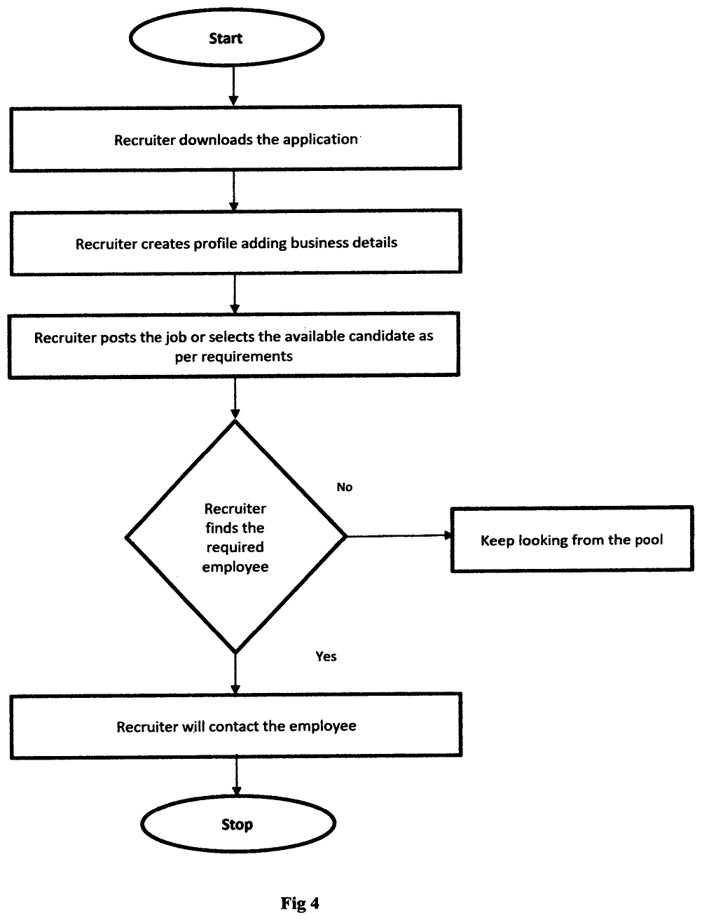

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The current invention in its preferred embodiment discloses an efficient system of selecting need based employees specifically for hospitality industry ranging from high-end restaurants, fast-food eateries, catering establishments and many other manifestations.

The system aims to provide an online platform for the recruiters which are basically from hospitality industry and they can place their on-demand staffing requirements using the disclosed application.

The invention as per its preferred embodiments allows to ensure that if someone is looking for job, is available for some additional shifts, might have lost the job or wants to switch over, the proposed guaranteed database of available job postings, posted by active employers are not be missed.

The system as per preferred embodiment consist of a landing screen once user downloads the application. There are 2 sets of profiles that can be created once application is downloaded. One is for job seekers and other is for recruiters. The user or candidate looking for job creates a profile allowing them to set member specific set of requirements and also to set their status as active/visible/available for work.

The candidate will be asked to create a profile which may requires but not limited to users' name, photo, city/state, Phone number, Email address, Date of birth, age, experience level, years of service, Certifications, licenses, availability, brief job history, POS experience, desired pay and background check agreement.

The job seekers are rated and displayed based on multiple indicators selected by the recruiter. This information is highly dynamic and can be updated as per recruiter's requirement.

The other set of profile creation is specifically addressed for the recruiters. The recruiter would download the app and create a profile including details lice name of business, type of business, address, Phone number, email address, business logo, business license, valid liquor license, services needed, hours of operation, uniform requirements, environment type and other free fields to set specific requirements.

The system as per its further embodiments allows that once the candidate has downloaded the app, successfully created their profile they'll be all set to go live for work. The candidate would login and will have an option to select their desired mode which can either be an active mode, busy mode or away mode. The active mode shows that the candidate is “visible” for work. They can go in and select the desired rate of pay and the travel radius. The candidate will be notified if there is any alert for them.

Once the candidate applies for a job or be alerted that their job request has been accepted the candidate receives an alert, they will then be given the option to chat via text with the employer (if needed). Thereafter a summary screen with the detail of the job, uniform requirements, pay rate, arrival time and on-site contact a appears. Once the candidate accepts the job a map (powered by Google) will appear and will route the candidate to the job site.

As per its further embodiment, once the time draws near the candidate will be alerted the best departure time based on their current location. The employer will also receive an alert advising the candidate is in route. Once the candidate arrives on site they will check-in on the app and after meeting with the employer briefly there will be an option to hit START to start the candidates pay clock.

If the candidate works more than 5 hours there should be a break given in which the candidate will have the option to clock-in/out for on the app. Once the candidates shift end they will have the option to reconnect with the point person and the candidate would then clock-out and been shown their total number of hours worked. The employer would then receive an alert to confirm the candidate's hours. Once confirmed the employer card on file will be charged and the candidates funds would be credited to their card on file.

Once approved the employer will be given the option to rate the candidate on a (star rating). This rating system is important for any candidate profile, because as this candidate goes out on jobs and accumulates good rating they will then become a “priority candidate” for that area and could at some point be given an increased pay rate.

The other interface which is for the recruiter. Once the recruiter has set up their profile the employer would have the option to select candidates within the desired radius and all active candidates in the area would appear on the Google powered map. The employer would then have a list of candidates appear to choose from based on the number of candidates “active” at the time of search.

Once the candidate accepts the job the employer will then have the ability to track the candidate when they're in route to the job. Once the candidate arrives the employer will be alerted through the app and the candidate's photo and name will appear on the screen.

The employer would give the candidate a brief tour and run down of the work space. Once that's done the candidate will press start shift and the employer will the alert and hit accept that will this start the time clock for the employee.

Should the candidate take any breaks they'll use the clock-in/clock out feature, and the employer will be notified of when the employee has done so in efforts to track and confirm all actual hours worked. Once job hours completed the employee would then clock out from their shift the employer will be alerted and the employer will verify the hours and hit accept and rate the level of service.

The system as per its additional embodiments differs from other similar service providers because of its rating system that is implemented. When developing the reviews of the clients, the focus is put on their individual strengths and weaknesses. The system focuses both on soft skills and hard skills. From the employer's reviews and experience this rating system is developed which allows other employers to engage the best available resources. This system of rating is equally beneficial for both parties allowing the candidate to have better pay rate and recruiters to select the best available resource as per their requirements.

The system also provides a server which can securely store the data and allow it to be utilized in effective manner. The storage services data can be shared by the authorized members with their new prospective employees to share their past references and ratings. This data can be shared either by download and email, or otherwise.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a singe disclosed embodiment. Thus, the following claims are hereby incorporated into the Detailed Description, with each claim standing on its own as a separately claimed subject matter.

We claim:

I: A computer-aided method of employing personnel for hospitality industry comprising:
(a) a website to display on the user's output screen,
(b) providing an end user interface on the user's device,
(c) providing said website for running on mobile devices, laptops or desktop screen,
(d) a user terminal
(d) means for collecting information from said end user by a display of one or more screens of selectable information at the beginning of execution of the application,
(e) whereby said website will store user's profile either in applicant or recruiter category;
(f) means for user to select their status of availability;
(g) recruiter can select applicant based on their requirements and applicants' past referrals;
(h) means for finalizing job details and timelines and
(i) means for the payment of dues once the job is completed.

II. The method of claim 1, wherein multiple indicators can be used by recruiters to select employees including but not limited to age, availability, past experiences, pay rate and others. The system further allows to leave feedback and rate the completed jobs and applicants to build the rating system facilitating the creation of true and realistic ratings.
